(19) United States
(12) Patent Application Publication
(10) Pub. No.: US 2013/0209441 A1
(43) Pub. Date: Aug. 15, 2013

(54) PROCESS FOR INHIBITION OF CEREBRAL DAMAGE ASSOCIATED WITH ISCHEMIA BY ANTHOCYANINS AND ANTHOCYANIDINS

(71) Applicants: Muraleedharan G. Nair, Okemos, MI (US); Jiangyong Min, East Lansing, MI (US); Arshad Majid, Bradford (GB)

(72) Inventors: Muraleedharan G. Nair, Okemos, MI (US); Jiangyong Min, East Lansing, MI (US); Arshad Majid, Bradford (GB)

(21) Appl. No.: 13/468,712

(22) Filed: May 10, 2012

Related U.S. Application Data
(60) Provisional application No. 61/598,843, filed on Feb. 14, 2012.

(51) Int. Cl. A61K 31/7048; A61P 7/02; A61K 38/49; A61K 31/352; A61P 25/00

(52) U.S. Cl. 424/94.64; 514/27; 514/456

(57) ABSTRACT

A process is provided for inhibition of neural damage associated with an ischemic event that includes the administration of anthocyanin compound to a subject. After allowing sufficient time for the anthocyanin compound to reach the situs of the ischemic event, inhibition of neural damage associated with the ischemic event occurs. Neural damage is further inhibited by administration of the anthocyanin compound in conjunction with an agent effective in and specifically including tissue plasminogen activator. The anthocyanin compound in specific embodiments is in pure form or a mixture of two or more anthocyanins, or anthocyanidins or aglycones thereof, with the mixture being a natural product extract.

A composition is also provided that includes ananthocyanin compound to cause blood clot dissolution. The agent being in specific embodiments is tissue plasminogen activator or aspirin.

### PRIORITY BENEFIT

This application is a non-provisional application that claims priority benefit of U.S. Provisional Application Ser. No. 61/598,843 filed Feb. 14, 2012; the contents of which are herein incorporated by reference.

### FIELD OF THE INVENTION

The present invention in general relates to a process for inhibiting injury associated with ischemia, and in particular to the use of anthocyanins and anthocyanidins in such a process.

### BACKGROUND OF THE INVENTION

Cerebral artery occlusion during stroke initiates a cascade of deleterious processes that contribute to brain injury. Increased generation of free oxygen radicals is one such process that leads to the activation of pro-apoptotic downstream signals [10]. Ischemia induced mitochondrial injury and the subsequent activation of pro-apoptotic proteins in mitochondria like cytochrome c (cyt c) and apoptosis-inducing factor (AIF), may enhance neuronal apoptotic death after ischemia [5,10].

The dipeptide carnosine has been shown to reduce brain damage during focal cerebral ischemia [8]. It is possible, therefore, that other natural antioxidants may have a neuroprotective effect against focal cerebral ischemia yet have more attractive dosing profiles. Anthocyanins are natural pigments abundant in tart cherries [9]. Anthocyanins are also present in beans, fruits, vegetables, and other edible plant materials. The anthocyanins, including cyanidin-3-O glycosides (CG), are strong antioxidants and also possess anti-inflammatory properties [14].

Thus, there exists a need for a natural product anthocyanin to be administered prior to, or subsequent to an ischemic event to inhibit a secondary injury associated with such an event.

### SUMMARY OF THE INVENTION

A process is provided for inhibition neural damage associated with an ischemic event that includes the administration of anthocyanin compound to a subject. After allowing sufficient time for the anthocyanin compound to reach the situs of the ischemic event, inhibition of neural damage associated with the ischemic event occurs. Neural damage is further inhibited by administration of the anthocyanin compound in conjunction with an agent effective in ischemia treatment and specifically including tissue plasminogen activator. The anthocyanin compound in specific embodiments is in pharmaceutically pure form or is a mixture of two or more anthocyanins, or anthocyanidins or aglycones thereof The mixture being free of other active compounds or is a natural product extract.

A composition is also provided that includes an anthocyanin compound for blood clot dissolution. The agent being in specific embodiment is tissue plasminogen activator or aspirin.

### BRIEF DESCRIPTION OF THE FIGURES

FIGS. 1A-1D are plots of infarct volume (A and B) and neurological score (C and D) as a function of dose of cyanidin-3-O-glucoside (C3G) 24 hours post middle cerebral artery occlusion (pMCAO) (A and C) against a vehicle control, effects of pre-treatment versus post-treatment are also shown (B and D);

FIGS. 2A-2C are confocal micrographs of mouse brain sections of (A) sham-operated, (B) vehicle-treated, and (C) 2 mg/kg of C3G-treated pMCAO mice; FIG. 2D are bar graphs represent the average superoxide intensity of fluorescence from pMCAO mice at 24 h after ischemic stroke and treatment of C3G, n=4 in each group; and

FIGS. 3A-3E are electrophoretic gels showing cytochrome C or AIF release in situ from isolated mitochondria from adult mouse brains. (A) Cyt c release in response to Ca²⁺ (100 μM) stimulation in the presence of 1 μM or 100 μM of C3G, respectively. (B) Cyt c release in response to GS—NO (50 μM) oxidative stress in the presence of 100 μM of C3G. (C) Cyt c release in response to H₂O₂ (50 μM) oxidative stress in the presence of 100 μM of C3G. (D) AIF release in response to GS—NO (50 μM) stimulation in the presence of 100 μM of C3G. (E) AIF release in response to H₂O₂ (50 μM) stimulation in the presence of 100 μM of C3G. These results indicate that C3G has no effect on cyt c release. However, C3G inhibits AIF release from isolated mitochondria.

### DETAILED DESCRIPTIOIN OF THE PREFERRED EMBODIMENTS

The present invention has utility in limiting cerebral damage or damage induced in nervous system tissue by ischemia. While the present invention is detailed hereafter with respect to the anthocyanin cyanidin-3-O-glucoside (C3G), it is appreciated that these results extend to other anthocyanins, anthocyanidins, and aglycones thereof; these other compounds illustratively including pelargonidin, cyanidin, delphinidin, malvidin, peonidin, zebrinin, and those other anthocyanins recited in Harborne, J. B., Grayer, R. J. (1988) The Anthocyanins. In The Flavonoids. Ed. J. B. Harborne. London: Chapman and Hill pp. 1-20. The present invention provides for the administration prior to, or subsequent to an ischemic event to limit damage to brain tissue induced by the ischemic event.

As used herein, the term “ischemia”, and the related term ischemic event, denote clinical presentation of global ischemia, myocardial ischemia, subarachnoid hemorrhage, intracerebral hemorrhage, small vessel ischemic disease, and stroke; and an occurrence of ischemia, respectively.

As used herein, the term “administering” illustratively includes delivery of an anthocyanin to a subject by a route illustratively including systemic administration, local administration, injection, intravitreal injection, subconjuctival injection, sub-tenon injection, retrobulbar injection, suprachoroidal injection, surgical implantation, topical administration, iontophoretic delivery, oral, rectal, parenteral, intravenous, intramuscular, subcutaneous, intracisternal, intravaginal, intraperitoneal, intravesical, intraventricular, intracranial, intratumoral, local, transdermal, intrabuccal, intranasal, intrathecal, modifications thereof, or combinations thereof.

As used herein an “anthocyanin” compound is an anthocyanin, or an anthocyanidin each of which having a sugar moiety extending therefrom, or an aglycone thereof; a sugar moiety illustratively includes as a glucosyl, a galactosyl, a rutinosyl, an arabinosyl, other hexosyls, other pentosyls, fructosyls or a combination thereof that induces a neuroprotective effect after an ischemic event of greater than 10% relative to a solution control in a mouse model per Example 1 hereafter.

An anthocyanin compound administered herein is optionally supplemented with one or more agents effective in reducing inflammation, reducing cerebral pressure, having a neuroprotective effect on cells, reducing excitotoxicity, reducing inflammation or dissolving blood clots. It has been surprisingly found that the antioxidant properties of the anthocyanin compound, according to the present invention, appears to improve the efficacy of conventional ischemic therapeutics. Without intending to be bound to a particular theory, it is believed that oxidizing radicals damage blood vessels thereby causing vessel inflammation and vessel inelasticity. An anthocyanin compound appears to speed the action of conventional ischemic therapeutics thereby more quickly restoring blood flow as a mode of inhibiting neural damage associated with the ischemic event. Examples of co-administered agents with an anthocyanin compound illustratively include small molecules such as choline magnesium salicylate, aspirin, dipyridamole, clopidogrel, warfarin, tissue plasminogen activator (tPA), and combinations thereof to restore blood flow. Aspirin, tPA, and the combination thereof represent clinically proven agents for use in conjunction with an anthocyanin compound.

As used herein, “pharmaceutically pure” or synonymously “pure” or purified” anthocyanin is defined as containing more than 90% of the total anthocyanin being a single compound by weight and independent of various adjuvants or carriers that are added thereto to facilitate administration or storage.

In a preferred embodiment, an anthocyanin compound is administered to a subject in an amount of between 0.1 and 50 milligrams per kilogram body weight. It is appreciated that the route of administration significantly impacts the needed dosage with intrathecal anthocyanin administration requiring far less anthocyanin compound than administration via routes that require crossing of epithelial layer into the blood stream and further routes that require the anthocyanin compounds to transit the blood-brain barrier. The doses of such agents in concert with an anthocyanin are the same therapy as conventional agent dosing with such agents.

An anthocyanin compound is readily delivered in concert with a pharmaceutically acceptable adjuvant, a pharmaceutically acceptable carrier or a combination thereof. The compounds of the present invention can be administered to a patient either alone or as part of a pharmaceutical composition. The inventive compositions are suitable for administration to patients by a variety of routes including intrathecally, intraventricularly, intravenously, orally, parenterally, and mucosally.

Compositions suitable for delivery illustratively include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers; diluents; solvents; or vehicles include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of the required particle size in the case of dispersions, and by the use of surfactants.

Compositions suitable for injection optionally include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of an injectable pharmaceutical form can be brought about by the use of agents delaying absorption, for example, aluminum monostearate and gelatin.

Solid dosage forms for oral administration include capsules, tablets, pills, powders, and granules. In such solid dosage forms, an inventive conjugate is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied with to a thickness such that the entire coating does not dissolve in the gastrointestinal fluids at pH below 5 associated with stomach acids, yet dissolves above pH 5 in the small intestine environment. It is expected that any anionic polymer exhibiting a pH-dependent solubility profile is readily used as an enteric coating in the practice of the present invention to achieve delivery of the active to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particularly preferred enteric coating material for use herein are those acrylic acid polymers and copolymers available under the trade name EUDRAGIT®, Roehm Pharma (Germany). The EUDRAGIT® series L, L-30D and S copolymers are most preferred since these are insoluble in stomach and dissolve in the intestine.

The enteric coating provides for controlled release of the active agent, such that release is accomplished at a predictable location in the lower intestinal tract below the point at which drug release would occur absent the enteric coating. The enteric coating also prevents exposure of the active agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated solid dosages of the present invention allow optimization of drug absorption, active agent protection, and safety. Multiple enteric coatings targeted to release the active agent at various regions in the lower gastrointestinal tract would enable even more effective and sustained improved delivery throughout the lower gastrointestinal tract.

The enteric coating optionally contains a plasticizer to prevent the formation of pores and cracks that allow the penetration of the gastric fluids into the solid dosage. Suitable plasticizers illustratively include, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The enteric coating is applied to a solid dosage using conventional coating methods and equipment. For example, an enteric coating can be applied to a solid dosage using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6.sup.th Ed. (Media, Pa.: Williams & Wilkins, 1995).

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to an inventive conjugate, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Optionally, an anthocyanin compound is co-administered with a marker. The administration of the marker provides the advantage of monitoring the function of the inventive process on neural cells or in a subject to whom the anthocyanin compound is administered. Markers operative herein illustratively include green fluorescent protein, luciferase, 13-galactosidase. It is appreciated that other suitable markers known in the art are similarly operative herein. A marker is illustratively radioactive, luminescent, biologically active, magnetically active so as to be discernible in vivo by magnetic resonance imaging (MRI), or otherwise amenable to detection by conventional neuro-scanning techniques known in the art. In specific embodiments, a marker is chelated or covalently bonded to the anthocyanin compound.

Various aspects of the present invention are illustrated by the following nonlimiting examples. The examples are for illustrative purposes and are not intended to be a limitation upon the practice of the present invention.

### EXAMPLE 1

Oxidative stress is one of the major processes that lead to neuronal damage after focal ischemia [5]. Cyanidin-3-O-glucoside (C3G) from tart cherry extract has been shown to have a protective effect in various disease models based on its antioxidant and anti-inflammatory properties [14]. Both pretreatment and delayed therapy with C3G significantly reduced cerebral infarct size in mice at 24 h following pMCAO and also improved neurological functional outcome. The delayed treatment benefits of C3G have clinical significance. Our results also show that C3G significantly decreased the brain levels of superoxide 24 h after focal ischemia, which is consistent with previous observations in other organs [12, 13]. Without intending to be bound to a particular theory, it is believed that reduction of superoxide could be one possible mechanism of action of C3G in reducing ischemic injury. Mitochondria are the targets for many intracellular anti-apoptotic and pro-apoptotic signals, which contribute to brain injury after ischemic stroke [5,10]. Deprivation of energy after pMCAO impairs mitochondrial permeability and releases factors promoting apoptotic cell death. In response to these pro-apoptotic signals, cyt c is released from the intermembrane space and triggers further downstream caspase-mediated apoptotic events. C3G did not inhibit cyt c release, which suggests that it may influence a cyt c independent pathway that leads to cell apoptosis/death. AIF is a flavoprotein that is released from the mitochondria after focal ischemia [11, 15]. It is normally confined to the mitochondria but translocates to the nucleus when apoptosis occurs [11]. AIF migration to the nucleus induces DNA fragmentation and apoptosis by a caspase-independent mechanism [7]. Oxidative stress-induced AIF release from mitochondria was partially blocked by C3G.

C3G is neuroprotective in a mouse model of pMCAO even when delivered 3 h after the onset of ischemia, which is a clinically relevant time point in stroke. CG decreased cerebral superoxide levels, inhibited AIF release from mitochondria, but did not influence the cyt c-related cell death pathway.

C3G is isolated from Prunus cerasus fruits as previously described [9] is dissolved in 0.9% normal saline (NS) and is administered by oral gavage to mice at 1-h before induction of pMCAO. Mice are randomly distributed into 2 groups and treated with vehicle (n=10), or 1 mg/kg (n=8), 2 mg/kg (n=10), and 5 mg/kg (n=9) of C3G. The doses chosen in the present study are based on previous reports [2]. Animals are euthanized at 24 h following pMCAO and infarct volumes are determined.

### EXAMPLE 2

To evaluate the effect of a delayed treatment at 3 h following pMCAO, mice are randomly assigned to vehicle (n=11), or 2 mg/kg of C3G administered orally at 3 h after pMCAO supplemented by two additional doses of 2 mg/kg of C3G 3 h apart. The dose of 2 mg/kg was chosen based on the results from Example 1, in which it is demonstrated that 2 mg/kg given orally showed equivalent efficacy on neuroprotective effects as 5 mg/kg. Animals are euthanized at 24 h following pMCAO and infarct volumes and neurological outcomes are determined.

Neurological assessment of experimental mice is performed according to an 18 point-based scale [1] at 1 h before induction of pMCAO and at 24 h following surgery. Brains are immediately removed following neurological assessment, rinsed in cold saline solution, cut into 1-mm-thick coronal sections, and processed for 2% 2,3,5-triphenylterazolium chloride (TTC) staining as previously described [6,8]. The infarct volumes from individual sections are then summed to determine the total brain infarct volume and adjusted for edema (corrected infarct volume).

Measurement of superoxide is performed in situ by superoxide-selective oxidative fluorescent dye dihydroethidium cell membranes (DHE) as previously described in [3]. DHE is freely permeable across cell membranes and fluoresces red when oxidized to ethidium bromide by superoxide. Unfixed frozen brain sections (30 um) are placed on glass slides and submerged in 10⁻⁶ mol/L DHE (Sigma) in phosphate buffered saline (PBS) buffer, pH 7.4 and incubated at 37° C. for 30 min in a dark humidified container. Fluorescence in ischemic regions of brain sections is then detected by a Zeiss 210 confocal microscope with a 590-nn long-pass filter. The intensity of the fluorescence is analyzed and quantified by densitometry (NIH Image software, version 1.37). The study included three groups (sham-operated, pMCAO with vehicle-treated, and pMCAO with 2 mg/kg of CG-treated mice; (n=4). Four brain sections of each mouse and five fields in each section are measured to calculate mean levels of the brain superoxide. Mitochondria are prepared from adult mouse brains (6-8 weeks old) by using the Percoll gradient method, as previously described [4]. After decapitation, the brain is quickly removed and washed in the isolation buffer (300 mM sucrose, 0.1 mM EGTA, 10 mM HEPES, pH 7.4). The brain is homogenized in 15 ml of buffer by using hand-held glass Potter-Elvehjem homogenizer with PTFE pestle. After 5 strokes, the cell debris and nuclei are centrifuged at 1330×g for 5 min. The supernatant is further centrifuged at 21,200×g for 10 min. Mitochondrial fraction is resuspended in 15% Percoll solution diluted in the isolation buffer and layered on top of a discontinuous Percoll gradient of 25/40%. The density gradient is centrifuged at 30,700×g for 10 min and mitochondria are collected from the interface between 25 and 40% Percoll solution, transferred to a new tube, and washed in 10 mL of isolation buffer by centrifugation at 6700×g for 10 min. The resulting pellet is suspended in the isolation buffer without EGTA and further diluted to 2 mg/mL in assay buffer for the cyt c release assay. 50 μL of mitochondria are then mixed with buffer or reagents in a final reaction volume of 100 L.

The assay buffer contains 125 mM KCI, 2 mM KH₂PO₄, 4 mM MgCl₂ at pH 7.4, and 3 mM ATP, 0.8 mM ADP, 5 mM succinate and 2 μM rotenone for respiration. Mitochondrial suspensions diluted in the assay buffer are exposed to Ca²⁺ (100 μM), S-nitrosoglutathione (GS—NO, 50 μM for nitric oxide generation) or H₂O₂ (50 μM for oxidative stress) in 10 min and centrifuged sat 12,000×g in 5 min. The supernatant is collected and 15 μL of aliquots are run on a SDS-PAGE gel to detect the release of cyt c by the immunoblotting assay. To examine the effect of C3G on cyt c release, C3G (100 μM in assay buffer) or buffer only is added to prepared mitochondria 5 min before starting the assay and kept in the reaction mixture during the assay.

Mitochondria are prepared from mouse brains using flowing differential centrifugation procedures at 4° C. described previously [15]. Homogenation buffer (50 μl) containing the prepared mitochondrial pellet is incubated with 25 μl of C3G at the desired concentration and the buffer added to achieve the final volume of 100 μl. The AIF release is then assessed in the presence of 100 μM of C3G in response to stimulation by GS-NO (50 μM) and H₂O₂ (50 μM).

All data are expressed as mean values±S.E.M. The degree of statistical significance between groups is determined on the basis of Student t-test, One-way ANOVA test, followed by post hoc Fisher LSD test, and Kruskal-Wallis rank test. SigmaStat 3.0 software (SYSTAT Software Inc., Point Richmond, Calif.) is used to carry out the analysis. Statistical significance is defined at p<0.05.

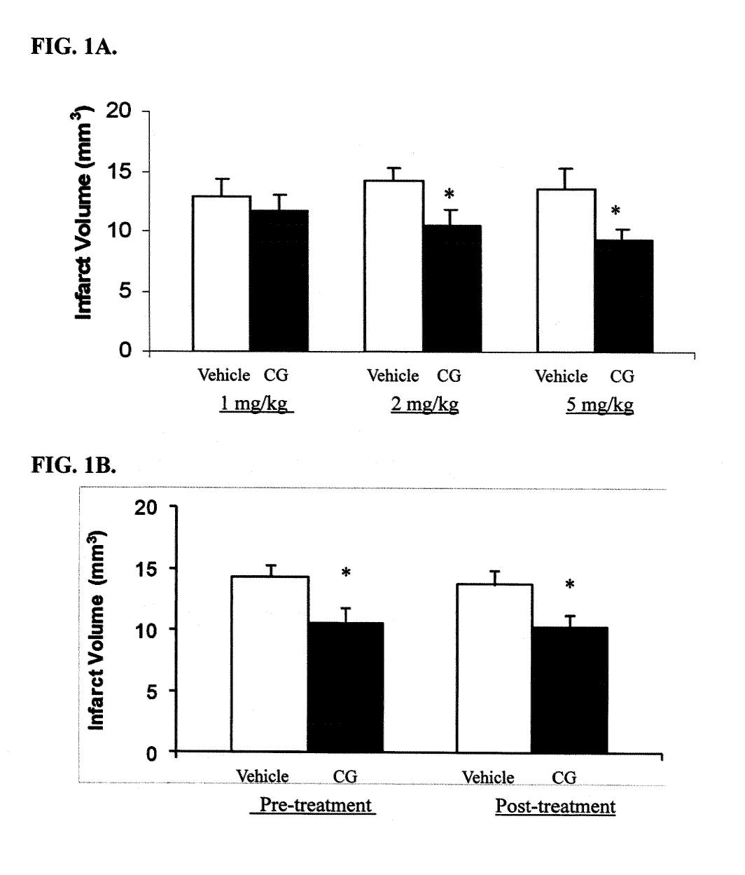

Physiological parameters are recorded in all experimental mice during surgical procedure. A significant reduction in regional cerebral blood flow (CBF) is observed in the lesioned cerebral hemisphere in ischemic mice following pMCAO. However, no differences are observed in the baseline CBF and CBF reduction after pMCAO. No differences in rectal temperature among experimental groups are noted (Table 1). Twenty four hours following pMCAO, C3G pretreatment, TTC staining shows reduced infarct size in a dose-dependent manner, as shown in FIG. 1A.

Effects of C3G on infarct size and neurological function in mice at 24 h after pMCAO. (A) Quantification of the infarct volume (mm³) shows a dose-dependent effect. The bar graph shows 2 mg/kg and 5 mg/kg of C3G provide significant reduction in infarct size. (B) Delayed treatment with C3G provides similar effects on reduction of infarct volume. (C) Neurological function is significantly better in mice treated with 2 mg/kg and 5 mg/kg. (D) Both pre- and post-treatment with C3G results in better functional outcome assessed by neurological scores in mice at 24 h following pMCAO. Histogram values represent means±S.E.M. (*p<0.05 C3G treated mice vs. the same batch of experimental mice with vehicle treatment).

Both 2 mg/kg and 5 mg/kg of C3G significantly reduces infarct volume by 27% and 30%, respectively. Interestingly and more relevant to clinical stroke, delayed treatment of C3G at 3 h following pMCAO supplemented with two additional doses of C3G 3 h apart also resulted in a 25% significant reduction in infarct size, as shown in FIG. 1B.

Neurological scores are normal (18 points) in all vehicle- and drug-treated mice before onset of pMCAO. Twenty-four hours following surgery, vehicle-treated mice exhibit a significant decrease in their neurological scores. In contrast, pretreatment with 2 or 5 mg/kg of C3G results in statistically significant better neurological performance compared to vehicle-treated mice (FIG. 1C). Neurological functional performance was also significantly better in pMCAO mice treated with C3G at 3 h after the onset of ischemia, as shown in FIG. 1D.

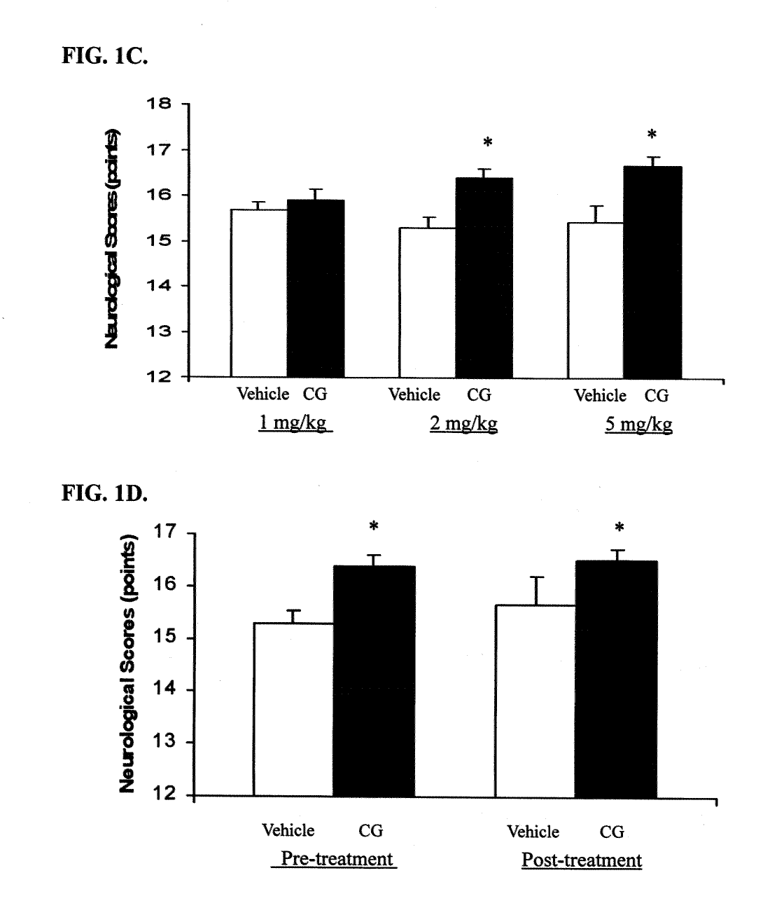

In situ detection of superoxide (0.1-mm anterior and 2-mm posterior to Bregma) reveals that the DHE fluorescent densities were more robust in brains from vehicle-treated groups compared to sham-operated mice, as shown in FIGS. 2A and B.

The results indicate that the level of brain superoxide in vehicle-treated mice compared to sham-operated mice. Treatment with C3G significantly decreased the brain level of superoxide compared to vehicle-treated mice. C3G, cyanidin-3-O-glucoside treated mice at dose of 2 mg/kg orally. *p<0.05 Vehicle vs Sham; *p<0.05 C3G vs Vehicle. Pretreatment with C3G (2 mg/kg) significantly suppressed the fluorescent density of superoxide in situ, as shown in FIG. 2C. Quantitative analysis indicates that levels of superoxide fluorescent density are significantly lower in C3G treated mice compared to the vehicle-treated group, as shown in FIG. 2D.

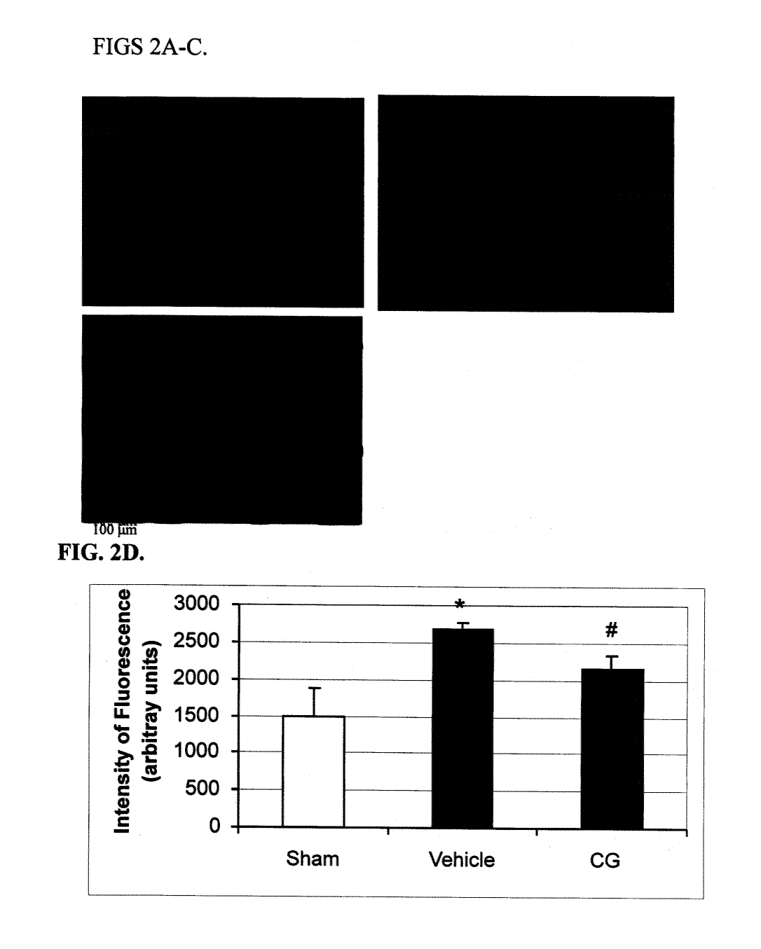

Previous reports have demonstrated that Ca²⁺ and oxidative stress to mitochondria results in cell death and brain tissue damage after ischemia [10]. Release of cyt c from mitochondria may potentiate cellular injury. To test whether C3G could prevent the release of cyt c, we examined the release of cyt c by stimulation of Ca²⁺ and oxidative stressors (GS-NO and H₂O₂) in isolated mouse brain mitochondria in situ. As demonstrated in FIG. 3A-C, immunoblot analysis of the supernatant of mitochondria showed increased release of cyt c following exposure to Ca²⁺, GS—NO, or H₂O₂. However, addition of C3G (100 uM) did not prevent the release of cyt c. In contrast, as shown in FIGS. 3D and E, although there was increased release of AIF in the presence of GS—NO or H₂O₂, addition of C3G (100 μM) partially prevented AIF release triggered by both GS—NO and H₂O₂ stimulations.

Cyt c or AIF released in situ from isolated mitochondria from adult mouse brains. In FIG. 3(A) Cyt c release in response to Ca²⁺ (100 μM) stimulation in the presence of 1 μM or 100 μM of C3G is shown, respectively. In FIG. 3(B) Cyt c release in response to GS—NO (50 μM) oxidative stress in the presence of 100 μM of C3G is shown. In FIG. 3(C) Cyt c release in response to H₂O₂ (50 μM) oxidative stress in the presence of μM of C3G is shown. In FIG. 3(D) AIF release in response to GS—NO (50 μM) stimulation in the presence of 100 μM of C3G is shown. In FIG. 3(E), AIF release in response to H₂O₂ (50 μM) stimulation in the presence of 100 μM of C3G is shown. These results indicate that C3G has no effect on cyt c release. However, inhibits AIF release from isolated mitochondria.

### EXAMPLE 3

The process of Example 2 is repeated with pelargonidin with similar effect against pMCAO.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof The following claims, including all equivalents thereof, are intended to define the scope of the invention.

The following references are each incorporated herein by reference for the entirety of their contents as if each reference were fully and explicitly included.

### EXAMPLE 4

The process of Example 2 is repeated with a natural product extract from tart cherry fruits obtained by the process detailed in reference 2, 9 and 14. The natural product extract including 65% cyanidin-3-O-glucosyl rutinoside, 30% cyaniding-3-O-rutinoside, and _{—}5% cyaniding-3-O-glucoside where percentages represent the percentage of anthocyanin compounds present in the natural extract and where the total anthocyanin compound loading within the natural extract is >50% with the remainder of the natural product extract including anthocyanidins and other natural products including phenolics. The natural product extract provided similar effect against pMCAO relative to the results obtained in Example 2.

### REFERENCES

[1] J. Garcia, S. Wagner, K. F. Liu, X. J. Hu, Neurological deficit and extent of neuronal necrosis attributable to middle cerebral artery occlusion in rats: statistical validation, Stroke 26 (1995) 627-635.
[2] B. Jayaprakasarn, L. K. Olson, R. F. Schutzki, M. H. Tai, M. G. Nair, Amelioration of obesity and glucose intolerance in high-fat-fed C57BI/G mice by anthocyanins and ursolic acid in cornelian cherry (Cornus mus), J. Agric. Food Chem. 54 (2006) 243-248.
[3] G. A. Kidd, H. Hua, A. Majid, D. Kaufman, A. F. Chen, Inhibition of brain GTP cyclohydrolase I and tetrahydrobioproterin attenuates cerebral infarction via reducing inducible NO synthase and peroxynitrite in ischemic stroke, Stroke 36 (2005) 2705-2711.
[4] T. Kristian, G. Fiskum, A fluorescence-based technique for screening compounds that protect against damage to brain mitochondria, Brain Res. Protoc. 3 (2000) 176-182.
[5] E. H. Lo, T. Dalkara, M. A. Moskowitz, Mechanisms, challenges and opportunities, Stroke. Nat. Res. 4 (2003) 399-415.
[6] J. Min, M. U. Farooq, E. Greenberg, F. Aloka, A. Blatt, M. Kassah, J. P. Morgan, A. Majid, Cardiac dysfunction after left permanent cerebral focal ischemia: the brain and heart connection, Stroke 40 (2009) 2560-2563.
[7] J. M. Penninger, G. Kroemer, Mitochondria, AIF, and caspases-rivaling for cell death execution, Nat. Cell Biol. 5 (2003) 97-99.
[8] G. K. Rajanikant, D. Zemke, M. C. Senot, M. B. Frenkel, A. F. Chen, R. Gupta, A. Majid, Carnosine is neuroprotective against permanent focal cerebral ischemia in mice, Stroke 38 (2007) 3023-3031.
[9] N. P. Seeram, R. Schutzki, A. Chandra, M. G. Nair, Characterization, quantification, and bioactivities of anthocyanins in Cormus species, J. Agric. Food Chem. 50 (2005) 2519-2523.
[10] N. R. Sims, M. F. Anderson, Mitochondrial contributions to tissue damage in stroke, Neurochem. Int. 40 (2000) 511-526.
[11] S. A. Susin, H. K. Lorenzo, N. Zamzami, I. Marzo, B. E. Snow, G. M. Brothers, J. Mangion, E. Jacotot, P. Constantini, M. Loeffer, N. Larochette, D. R. Goodlett, R. Aebersold, D. P. Siderovski, J. M. Penninger, G. Kroemer, Molecular characterization of mitochondrial apoptosis-inducing factor, Nature 379 (1999) 441-445.
[12] M. C. Toufeksian, M. de Loregeril, N. Nagy, P. Salen, M. B. Donati, J. Giordano, H. P. Mock, S. Peterek, A. Matros, K. Petroni, R. Pilu, D. Rotilio, C. Tonelli, J. de Leiris, F. Boucher, C. Martin, Chronic dietary intake of plant-derived anthocyanins protects the rat heart against ischemia-reperfusion injury, J. Nutr. 138 (2008) 747-752.
[13] T. Tsuda, F. Horio, T. Oszwa, Protective effects of dietary cyaniding 3-0 betagluroside on liver ischemia-reperfusion injury in rats, Arch. Biochem. Biophys. 368 (1999) 361-366.
[14] H. Wang, M. G. Nair, G. M. Strasburg, Y. C. Chang, A. M. Booren, J. I. Gray, D. DeWitt, Antioxidant and anti-inflammatory activities of anthocyanins and their aglycon, cyanidin, from tart cherries, J. Nat. Prod. 62 (1999) 294-296.
[15] S. W. Yu, S. A. Andrabi, H. Wang, N. S. Kim, G. G. Poirier, T. M. Dawson, V. L. Dawson, Apoptosis-inducing factor mediates poly(ADP-ribose) (PAR) polymer-induced cell death, Proc. Natl. Acad. Sci. U.S.A. 103 (2006) 18314-18319.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process of inhibiting neural damage associated with an ischemic event in a subject comprising:
administering an anthocyanin compound to the subject; and
allowing sufficient time for said anthocyanin compound to inhibit the neural damage associated with the ischemic event.

2. The process of claim 1 wherein said administering of said anthocyanin compound is prior to the ischemic event.

3. The process of claim 1 wherein said administering of said anthocyanin compound is within 72 hours subsequent to the ischemic event.

4. The process of claim 1 wherein said administering of said anthocyanin compound is within 3 hours subsequent to the ischemic event.

5. The process of claim 1 wherein said administering of said anthocyanin compound is within 24 hours subsequent to the ischemic event.

6. The process of claim 1 wherein said administering of said anthocyanin compound is within 3 hours subsequent to the ischemic event.

7. The process of claims 1 further comprising:
co-administering an agent with said anthocyanin compound, said agent effective in at least one of reducing inflammation, reducing cerebral pressure, protecting neural cells, reducing excitotoxicity, reducing infection or blood clot dissolution.

8. The process of claim 6 wherein said agent is tissue plasminogen activator or aspirin.

9. The process of claims 1 wherein said anthocyanin compound is administered in an amount of between 0.1 and 50 mg per kilogram body weight of the subject.

10. The process of claims 1 wherein said administering is through a route or oral, intravenous, intracranial, or intrathecal.

11. The process of claims 1 wherein said anthocyanin compound comprises at least one cyanidin-3-O-hexose sugar.

12. The process of claims 1 wherein said anthocyanin compound comprises a cyanidin-3-O-glycoside as a majority by weight of said anthocyanin compound.

13. The process of claims 1 wherein said anthocyanin compound consists essentially of cyanidin-3-O-glucoside in purified form along with optional adjuvants or optional carriers.

14. A process of inhibiting neural damage associated with an ischemic event in a subject comprising:
administering an anthocyanin compound comprising a cyanidin-3-O-glycoside as the majority anthocyanin compound by weight and at least one of tissue plasminogen activator or aspiring; and
allowing sufficient time for said anthocyanin compound to inhibit the neural damage associated with the ischemic event.

15. The process of claim 14 wherein said administering is within 24 hours subsequent to the ischemic event.

16. The process of claim 14 wherein said anthocyanin compound is a mixture of two or more anthocyanins or anthocyanidins, or aglycones, said mixture being a natural product extract.

17. A composition comprising:
an anthocyanin compound; and
an agent effective in blood clot dissolution.

18. The composition of claim 17 further comprising: a carrier compatible with said anthocyanin compound and said agent.

19. The composition of claim 17 wherein said agent is tPA or aspirin.

20. The composition of claims 11 further comprising an adjuvant.
